(19) United States
(12) Patent Application Publication
(10) Pub. No.: US 2006/0074392 A1
(43) Pub. Date: Apr. 6, 2006

(54) Disposable menstrual underwear

(76) Inventor: Bernadette Jean Sava, Sherman Oaks, CA (US)

Correspondence Address: Bernadette Sava, #235, 5345 Sepulveda Blvd., Sherman Oaks, CA, 91411, US

(21) Appl. No.: 11/226,595

(22) Filed: Sep. 15, 2005

Related U.S. Application Data
(60) Provisional application No. 60/522,441, filed on Oct. 1, 2004.

(51) Int. Cl. A61F 13/15

(52) U.S. Cl. 604/402; 604/367

(57) ABSTRACT

A containment chamber to hold the menstrual fluid in which keeps the moisture away from the body built in to a pair of disposable underwear.

### RELATED APPLICATIONS

The present application is related to U.S. patent No. 60/522,441, issued Oct. 1, 2004, for DISPOSABLE MENSTRUAL UNDERWEAR, by Bernadette Sava, included by reference herein.

### FIELD OF THE INVENTION

The present invention relates to feminine hygiene products and, more particularly, to menstrual products.

### BACKGROUND OF THE INVENTION

Blood ruining underwear and other clothing from leakage of current products.

Soaking the clothing in bleach or other cleaning agents.

The bleach tends to break down the material or the products don't get the blood stain out and they have to be thrown away. This becomes very costly.

It is therefore an object of the invention to keep the moisture away from the body which is healthier for the woman.

It is another object of the invention to prevent the ruin of good clothing by supplying a product which is thrown away after each use.

It is another object of the invention to be constructed of biodegradable materials to help the environment.

It is another object of the invention to conform to the different postures of a woman better.

### SUMMARY OF THE INVENTION

In accordance with the present invention, there is provided a containment chamber to hold the menstrual fluid in which keeps the moisture away from the body built in to a pair of disposable underwear.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a front perspective view of a panty style being used;

FIG. 2 is a right detail view of a containment chamber breakdown;

FIG. 3 is a bottom section view of the containment chamber encasement can be constructed without side flap also;

FIG. 4 is a bottom detail view of the bamboo;

FIG. 5 is a front perspective view of a natural sponge which will fit inside the bamboo enclosure;

FIG. 6 is a top perspective view of a top of encasement with a ⅛″ opening;

FIG. 7 is a top section view of a silk material strips to leave an opening for the menstrual fluid to drip into containment chamber opening;

FIG. 8 is a right perspective view of the containment chamber and side flap sewn into panty can be constructed without side flap also;

FIG. 9 is a front detail view of a panty shaded to show area of containment chamber in version 1 and area of hollowed out pad in version 2;

FIG. 10 is a right section view of a hollowed out pad with side flap can be constructed without side flap also; and

FIG. 11 is a right perspective view of a hollowed out pad with side flap sewn into panty can be constructed without side flap also.

For purposes of clarity and brevity, like elements and components will bear the same designations and numbering throughout the Figures.

### DESCRIPTION OF THE PREFERRED EMBODIMENT

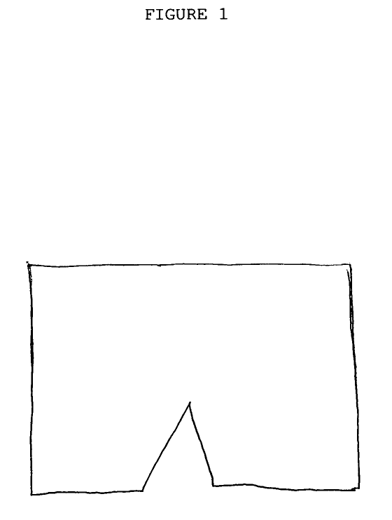

FIG. 1 is a front perspective view of a panty 10 style being used.

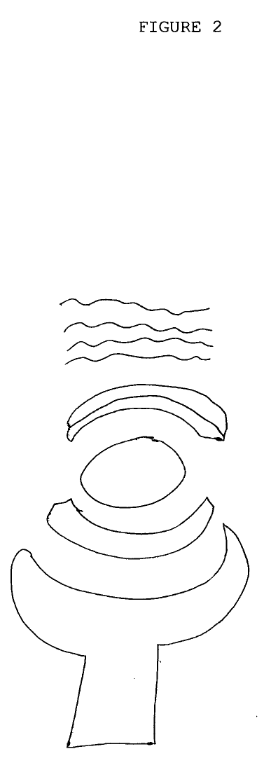

FIG. 2 is a right detail view of a containment chamber breakdown.

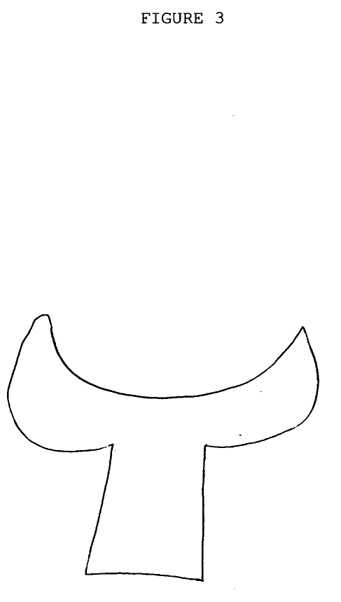

FIG. 3 is a bottom section view of the containment chamber encasement can be constructed without side flap also.

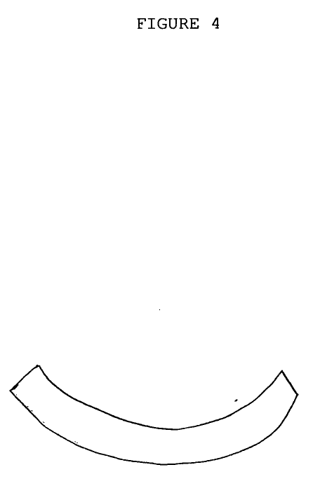

FIG. 4 is a bottom detail view of the bamboo 16.

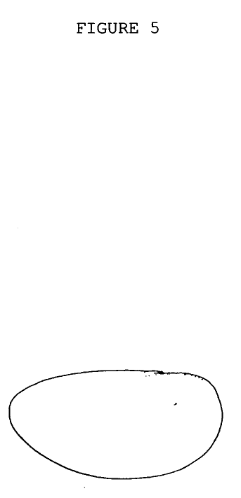

FIG. 5 is a front perspective view of a natural sponge 18 which will fit inside the bamboo 16 enclosure.

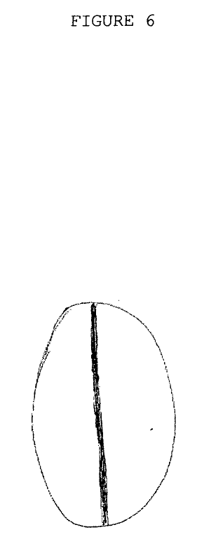

FIG. 6 is a top perspective view of a top of encasement with a ⅛″ opening.

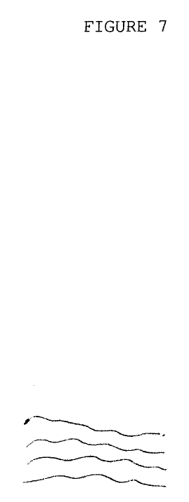

FIG. 7 is a top section view of a silk 22 material strips to leave an opening for the menstrual fluid to drip into containment chamber opening.

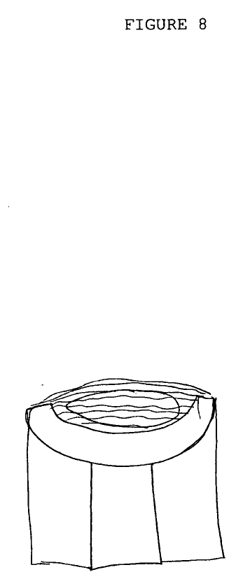

FIG. 8 is a right perspective view of the containment chamber and side flap sewn into panty 10 can be constructed without side flap also.

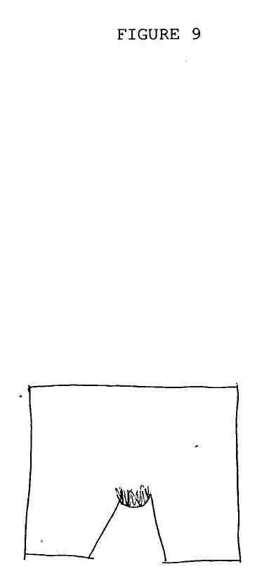

FIG. 9 is a front detail view of a panty 10 shaded to show area of containment chamber in version 1 and area of hollowed out pad in version 2.

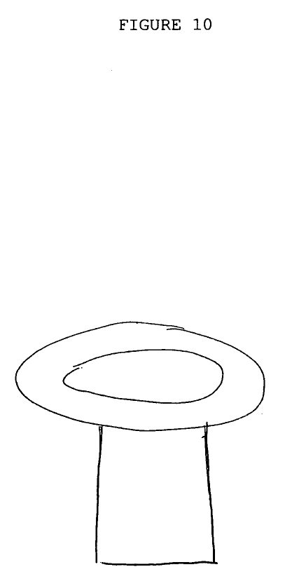

FIG. 10 is a right section view of a hollowed out pad with side flap can be constructed without side flap also.

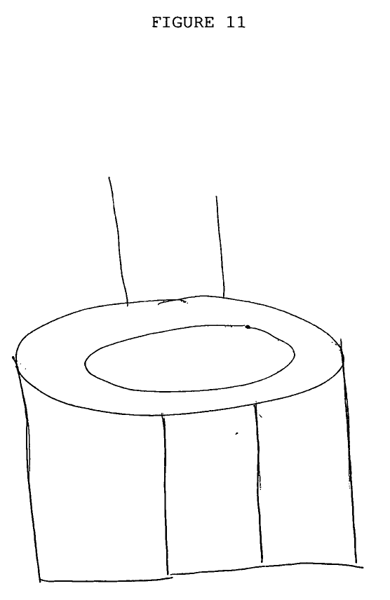

FIG. 11 is a right perspective view of a hollowed out pad with side flap sewn into panty 10 can be constructed without side flap also.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A new patent for disposable menstrual underwear, comprising:
means for holds the containment chamber in place and is what is worn and then disposed of;
means for holding the menstrual fluid in the containment chamber away from the body;
means for holding the containment chamber elements in place and adding protection from leakage;
means for holding sponge in place in a non-leaking reinforcement chamber, rigidly connected to said means for holding the containment chamber elements in place and adding protection from leakage;
means for catching and holding the menstrual fluid, rigidly connected to said means for holding sponge in place in a non-leaking reinforcement chamber; and
means for closing the encasement chamber leaving only an ⅛″ slit for the menstrual fluid to drip down into while trapping any spillage that may occur, rigidly connected to said means for holding the containment chamber elements in place and adding protection from leakage.

2. The new patent in accordance with claim 1, wherein said means for holds the containment chamber in place and is what is worn and then disposed of comprises a made of hemp which is biodegradable panty.

3. The new patent in accordance with claim 1, wherein said means for holding the menstrual fluid in the containment chamber away from the body comprises a biodegradable breakdown of containment chamber.

4. The new patent in accordance with claim 1, wherein said means for holding the containment chamber elements in place and adding protection from leakage comprises an unbleached cotton encasement with side flap.

5. The new patent in accordance with claim 1, wherein said means for holding sponge in place in a non-leaking reinforcement chamber comprises a bamboo.

6. The new patent in accordance with claim 1, wherein said means for catching and holding the menstrual fluid comprises a biodegradable natural sponge.

7. The new patent in accordance with claim 1, wherein said means for closing the encasement chamber leaving only an ⅛″ slit for the menstrual fluid to drip down into while trapping any spillage that may occur comprises a ⅛″ opening encasement closing.

8. A new patent for disposable menstrual underwear, comprising:
a made of hemp which is biodegradable panty, for holds the containment chamber in place and is what is worn and then disposed of;
a biodegradable breakdown of containment chamber, for holding the menstrual fluid in the containment chamber away from the body;
an unbleached cotton encasement with side flap, for holding the containment chamber elements in place and adding protection from leakage;
a bamboo, for holding sponge in place in a non-leaking reinforcement chamber, rigidly connected to said Encasement with side flap;
a biodegradable natural sponge, for catching and holding the menstrual fluid, rigidly connected to said Bamboo; and
a ⅛″ opening encasement closing, for closing the encasement chamber leaving only an ⅛″ slit for the menstrual fluid to drip down into while trapping any spillage that may occur, rigidly connected to said Encasement with side flap.

9. The new patent as recited in claim 8, further comprising:
a biodegradable silk, for a soft touch to the body, loosely connected to said Encasement closing.

10. A new patent for disposable menstrual underwear, comprising:
a made of hemp which is biodegradable panty, for holds the containment chamber in place and is what is worn and then disposed of;
a biodegradable breakdown of containment chamber, for holding the menstrual fluid in the containment chamber away from the body;
an unbleached cotton encasement with side flap, for holding the containment chamber elements in place and adding protection from leakage;
a bamboo, for holding sponge in place in a non-leaking reinforcement chamber, rigidly connected to said Encasement with side flap;
a biodegradable natural sponge, for catching and holding the menstrual fluid, rigidly connected to said Bamboo;
a ⅛″ opening encasement closing, for closing the encasement chamber leaving only an ⅛″ slit for the menstrual fluid to drip down into while trapping any spillage that may occur, rigidly connected to said Encasement with side flap; and
a biodegradable silk, for a soft touch to the body, loosely connected to said Encasement closing.
